(19) United States
(12) Patent Application Publication
(10) Pub. No.: US 2016/0151344 A1
(43) Pub. Date: Jun. 2, 2016

(54) COMPOUNDS FOR USE IN THE TREATMENT OF NEUROBLASTOMA, EWING'S SARCOMA OR RHABDOMYOSARCOMA

(71) Applicant: Xizhong Huang, Southborough, MA (US)

(72) Inventor: Xizhong Huang, Southborough, MA (US)

(73) Assignee: Novartis AG, Basel (CH)

(21) Appl. No.: 15/018,287

(22) Filed: Feb. 8, 2016

Related U.S. Application Data
(63) Continuation of application No. 14/387,636, filed on Sep. 24, 2014.
(60) Provisional application No. 61/618,215, filed on Mar. 30, 2012.

(51) Int. Cl. A61K 31/4439

(52) U.S. Cl. CPC A61K 31/4439

(57) ABSTRACT

The present invention relates to a method of treating cancer selected from the group consisting of neuroblastoma, Ewing's Sarcoma, or rhabdomyosarcoma comprising administering a therapeutically effective amount of a compound of formula (I), as defined herein, or a pharmaceutically acceptable salt thereof to a subject, preferably a human, in need thereof; to use of the compound of formula (I) or a pharmaceutically acceptable salt thereof for the manufacture of pharmaceutical compositions for use in the treatment of cancer selected from the group consisting of neuroblastoma, Ewing's Sarcoma, or rhabdomyosarcoma; and to use of the compound of formula (I) or a pharmaceutically acceptable salt thereof in the treatment of cancer selected from the group consisting of neuroblastoma, Ewing's Sarcoma, or rhabdomyosarcoma.

### FIELD OF THE INVENTION

The present invention relates to a method of treating cancer selected from the group consisting of neuroblastoma, Ewing's Sarcoma, or rhabdomyosarcoma comprising administering a therapeutically effective amount of a compound of formula (I), as described herein, or a pharmaceutically acceptable salt thereof to a subject, preferably a human, in need thereof; to use of the compound of formula (I) or a pharmaceutically acceptable salt thereof for the manufacture of pharmaceutical compositions for use in the treatment of cancers selected from the group consisting of neuroblastoma, Ewing's Sarcoma, or rhabdomyosarcoma; and to use of the compound of formula (I) or a pharmaceutically acceptable salt thereof in the treatment of cancers selected from the group consisting of neuroblastoma, Ewing's Sarcoma, or rhabdomyosarcoma.

### BACKGROUND OF THE INVENTION

Neuroblastoma is a disease in which malignant (cancer) cells form in nerve tissue of the adrenal gland, neck, chest, or spinal cord. Neuroblastoma is the most common extracranial childhood cancer and the most common tumor occurring during infancy. It is an embryonal malignancy of the sympathetic nervous system arising from neuroblasts (pluripotent sympathetic cells). In the developing embryo, these cells invaginate, migrate along the neuraxis, and populate the sympathetic ganglia, adrenal medulla, and other sites, e.g. malignant (cancer) cells form in nerve tissue of the adrenal gland, neck, chest, or spinal cord.

Neuroblastoma often begins in the nerve tissue of the adrenal glands. There are two adrenal glands, one on top of each kidney in the back of the upper abdomen. The adrenal glands produce important hormones that help control heart rate, blood pressure, blood sugar, and the way the body reacts to stress. Neuroblastoma may also begin in the chest, in nerve tissue near the spine in the neck, or in the spinal cord. Neuroblastoma most often begins during early childhood, usually in children younger than 5 years. It sometimes forms before birth but is usually found later, when the tumor begins to grow and cause symptoms.

Origin and migration pattern of neuroblasts during fetal development explains the multiple anatomic sites where these tumors occur; location of tumors appears to vary with age. Tumors can occur in the abdominal cavity (e.g. 40% adrenal, 25% paraspinal ganglia) or involve other sites (e.g. 15% thoracic, 5% pelvic, 3% cervical tumors, 12% miscellaneous).

By the time neuroblastoma is diagnosed, the cancer has usually metastasized (spread), most often to the lymph nodes, bones, bone marrow, liver, and skin. Most patients present with signs and symptoms related to tumor growth, although small tumors have been detected in infants on prenatal ultrasound. Large abdominal tumors often result in increased abdominal girth and other local symptoms (eg, pain). Paraspinal dumbbell tumors can extend into the spinal canal, impinge on the spinal cord, and cause neurologic dysfunction.

Ewing's Sarcoma is a rare and cancerous tumor that occurs in the bone or close to bone. Ewing's sarcoma typically affects children and adolescents, but usually develops during puberty. Ewing's Sarcoma is a group of four different types of cancer including of Ewing tumor of bone (Ewing sarcoma of bone), extraosseous Ewing tumors, primitive neuroectodermal tumors (peripheral neuroepithelioma), and Askin tumors. These tumors originate from the same type of stem cell.

Rhabdomyosarcoma is a rare and cancerous tumor of the muscles that are attached to bones. Most common locations for this tumor are the structures of the head and neck, urogenital tract and arms or legs. In the United States, there are approximately 350 cases of rhabdomyosarcoma diagnosed each year in children under the age of 21 years. a common soft tissue tumor in children.

Despite progress in medical treatment, improved treatment is still needed for patients suffering from neuroblastoma, Ewing's Sarcoma, and rhabdomyosarcoma.

According to the present invention, it has been found that the alpha-isoform specific phosphatidylinositol (PI) 3-kinase inhibitor compound of formula (I), particularly (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter “COMPOUND I”), or a pharmaceutically acceptable salt thereof are useful for effective treatment of neuroblastoma, Ewing's Sarcoma, and rhabdomyosarcoma.

### SUMMARY OF THE INVENTION

The present invention provides a method of treating cancer selected from the group consisting of neuroblastoma, Ewing's Sarcoma, or rhabdomyosarcoma comprising administering a therapeutically effective amount of a compound of formula (I), as defined herein, or a pharmaceutically acceptable salt thereof to a subject in need thereof.

A preferred compound of the present invention is a compound which is specifically described in WO2010/029082. A very preferred compound of the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter “COMPOUND I”) or a pharmaceutically acceptable salt thereof.

The present invention further provides a pharmaceutical composition or medicament comprising the compound of formula (I) or a pharmaceutically acceptable salt thereof, and optionally at least one pharmaceutically acceptable carrier, for use in the treatment of cancers selected from the group consisting of neuroblastoma, Ewing's Sarcoma, or rhabdomyosarcoma.

The present invention further provides a method of treating cancer selected from the group consisting of neuroblastoma, Ewing's Sarcoma, or rhabdomyosarcoma comprising administering a therapeutically effective amount of the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”) or a pharmaceutically acceptable salt thereof to a subject in need thereof.

The present invention further provides a method of delaying progression of cancers selected from the group consisting of neuroblastoma, Ewing's Sarcoma, or rhabdomyosarcoma in a patient comprising administering a therapeutically effective amount of a compound of formula (I) or a pharmaceutically acceptable salt thereof to a subject in need thereof.

The present invention further relates to the use of the compound of formula (I) or a pharmaceutically acceptable salt thereof for the manufacture of a pharmaceutical composition or medicament for use in the treatment of cancer selected from the group consisting of neuroblastoma, Ewing's Sarcoma, or rhabdomyosarcoma.

The present invention further relates to the use of the compound of formula (I) or a pharmaceutically acceptable salt thereof for the manufacture of a pharmaceutical composition or medicament for use in delaying the progression of cancers selected from the group consisting of neuroblastoma, Ewing's Sarcoma, or rhabdomyosarcoma in a patient in need thereof.

The present invention further relates to the use of the compound of formula (I) or a pharmaceutically acceptable salt thereof in the treatment of cancer selected from the group consisting of neuroblastoma, Ewing's Sarcoma, or rhabdomyosarcoma.

The present invention further relates to a compound of formula (I) or pharmaceutically acceptable salt thereof for use in the treatment of cancer selected from the group consisting of neuroblastoma, Ewing's Sarcoma, or rhabdomyosarcoma.

### SHORT DESCRIPTION OF THE FIGURES

FIG. 1 shows the sensitivity of neuroblastoma cell lines (CHP-212, IMR-32, KELLY, SK-N-SH, KP-N-SI9s, SIMA, SK-N-AS, SK-N-BE(2), SK-N-DZ, and SK-N-FI) upon treatment with COMPOUND I using the Two-Tailed Fisher's Exact Test. Two-Tailed Fisher's Exact Test provides a comparison of Amax (% inhibition) of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}amide) (“COMPOUND I”) to EC₅₀ of COMPOUND I.

FIG. 2 shows the sensitivity of Ewing's Sarcoma cell lines (HS 822.T, MHH-ES-1, SK-ES-1, and TC-71) upon treatment with COMPOUND I using the Two-Tailed Fisher's Exact Test. Two-Tailed Fisher's Exact Test provides a comparison of Amax (% inhibition) of COMPOUND I to EC₅₀ of COMPOUND I.

FIG. 3 shows the sensitivity of rhabdomyosarcoma cell lines (G401, G402, A673, Hs729, KYM-1, RD, RH-41, SJRH30, TE 125.T, and TE 617.T) upon treatment with COMPOUND I using the Two-Tailed Fisher's Exact Test. Two-Tailed Fisher's Exact Test provides a comparison of Amax (% inhibition) of COMPOUND I to EC₅₀ of COMPOUND I.

### DETAILED DESCRIPTION OF THE INVENTION

The present invention provides a method of treating cancer selected from the group consisting of neuroblastoma, Ewing's Sarcoma, or rhabdomyosarcoma comprising administering a therapeutically effective amount of a compound of formula (I), as defined herein, or a pharmaceutically acceptable salt thereof to a subject in need thereof.

Certain terms used herein are described below. Compounds the present invention are described using standard nomenclature. Unless defined otherwise, all technical and scientific terms used herein have the same meaning as is commonly understood by one of skill in the art to which this invention belongs.

The following general definitions shall apply in this specification, unless otherwise specified:

The terms “comprising” and “including” are used herein in their open-ended and nonlimiting sense unless otherwise noted.

The terms “a” and “an” and “the” and similar references in the context of describing the invention (especially in the context of the following claims) are to be construed to cover bot the singular and the plural, unless otherwise indicated herein or clearly contradicted by context. Where the plural form is used for compounds, salts, and the like, this is taken to mean also a single compound, salt, or the like.

The term “a phosphatidylinositol 3-kinase inhibitor” is defined herein to refer to a compound which targets, decreases or inhibits phosphatidylinositol 3-kinase (“PI3-kinase”). PI3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation.

The term “pharmaceutical composition” is defined herein to refer to a mixture or solution containing at least one active ingredient or therapeutic agent to be administered to a warm-blooded animal, e.g., a mammal or human, in order to prevent or treat a particular disease or condition affecting the warm-blooded animal.

The term “pharmaceutically acceptable” is defined herein to refer to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the warm-blooded animal in need thereof. Alternatively, a therapeutically effective amount is sufficient to cause an improvement in a clinically significant condition/symptom in the warm-blooded animal in need thereof.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

There are three classes of PI3-Kinases (I-III), and currently 8 members of the family are known. The class I enzymes consist of heterodimers having a regulatory (p85) domain and a catalytic (p110) subunit, of which there are four isoforms: p110α, p110β, p110δ and p110γ. The α and β isoforms are ubiquitously expressed; a is linked upstream mainly to receptor tyrosine kinases, whereas θ can mediate signals from both G-protein-coupled receptors and from receptor tyrosine kinases. The δ and γ isoforms are expressed primarily in lymphocytes and play important roles in the regulation of immune responses.

A gain of function in PI3K signaling is common in many types of human cancer and include inactivation of the PTEN tumor suppressor gene, amplification/overexpression or activating mutations of some receptor tyrosine kinases (e.g. erbB3, erbB2, EGFR), amplification of genomic regions containing AKT, amplification of PIK3CA (the gene encoding p110α) and mutations in p110α. More than 30% of various solid tumor types were recently found to contain mutations of PIK3CA. From these mutation frequencies, PIK3CA is one of the most commonly mutated genes identified in human cancers.

International Patent Application WO2010/029082 describes specific 2-carboxamide cycloamino urea derivatives, which have been found to have inhibitory activity for PI3-kinases (phosphatidylinositol 3-kinases). Specific 2-carboxamide cycloamino urea derivatives which are suitable for the present invention, their preparation and suitable formulations containing the same are described in WO2010/029082 and include compounds of formula (I)

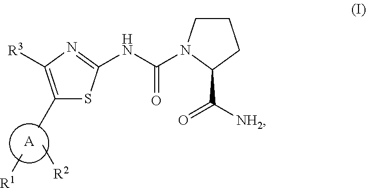

wherein
A represents a heteroaryl selected from the group consisting of:

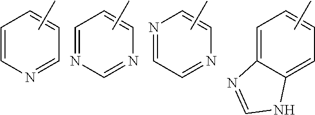

R¹ represents one of the following substituents: (1) unsubstituted or substituted, preferably substituted C₁-C₇-alkyl, wherein said substituents are independently selected from one or more, preferably one to nine of the following moieties: deuterium, fluoro, or one to two of the following moieties C₃-C₅-cycloalkyl; (2) optionally substituted C₃-C₅-cycloalkyl wherein said substituents are independently selected from one or more, preferably one to four of the following moieties: deuterium, C₁-C₄-alkyl (preferably methyl), fluoro, cyano, aminocarbonyl; (3) optionally substituted phenyl wherein said substituents are independently selected from one or more, preferably one to two of the following moieties: deuterium, halo, cyano, di(C₁-C₇-alkyl)amino, C₁-C₇-alkylaminocarbonyl, di(C₁-C₇-alkyl)aminocarbonyl, C₁-C₇-alkoxy; (4) optionally mono- or di-substituted amine; wherein said substituents are independently selected from the following moieties: deuterium, C₁-C₇-alkyl (which is unsubstituted or substituted by one or more substituents selected from the group of deuterium, fluoro, chloro, hydroxy), phenylsulfonyl (which is unsubstituted or substituted by one or more, preferably one, C₁-C₇-alkyl, C₁-C₇-alkoxy, di(C₁-C₇-alkyl)amino-C₁-C₇-alkoxy); (S) substituted sulfonyl; wherein said substituent is selected from the following moieties: C₁-C₇-alkyl (which is unsubstituted or substituted by one or more substituents selected from the group of deuterium, fluoro), pyrrolidine, (which is unsubstituted or substituted by one or more substituents selected from the group of deuterium, hydroxy, oxo; particularly one oxo); (6) fluoro, chloro;
R² represents hydrogen;
R³ represents (1) hydrogen, (2) fluoro, chloro, (3) optionally substituted methyl, wherein said substituents are independently selected from one or more, preferably one to three of the following moieties: deuterium, fluoro, chloro, dimethylamino;
with the exception of (S)-Pyrrolidine-1,2-dicarboxylic acid 2-amide 1-({5-[2-(tert-butyl)pyrimidin-4-yl]-4-methyl-thiazol-2-yl}-amide).

As disclosed in WO2010/029082, the compounds of formula (I) have been found to have particularly advantageous pharmacological properties and show an improved selectivity for the PI3-kinase alpha subtype as compared to other types.

The radicals and symbols as used in the definition of a compound of formula (I) have the meanings as disclosed in WO2010/029082 which publication is hereby incorporated into the present application by reference in its entirety.

A preferred compound of the present invention is a compound which is specifically described in WO2010/029082. A very preferred compound of the present invention is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (hereinafter “COMPOUND I”) or a pharmaceutically acceptable salt thereof. The synthesis of (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) is described in WO2010/029082 as Example 15.

The compounds of formula (I) may be used in form of the free base or a pharmaceutically acceptable salt thereof. The term “salts” (which, what is meant by “or salts thereof” or “or a salt thereof”), can be present alone or in mixture with free compound of the formula (I) and are preferably pharmaceutically acceptable salts. Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free cornpounds hereinbefore and hereinafter is to be understood as referring also to the corresponding salts, as appropriate and expedient. The salts of compounds of formula (I) are preferably pharmaceutically acceptable salts; suitable counter-ions forming pharmaceutically acceptable salts are known in the field.

In accordance with present invention, the compounds of formula (I) or a pharmaceutically acceptable salt thereof may be used to treat cancer selected from the group consisting of neuroblastoma, Ewing's sarcoma, or rhabdomyosarcoma.

Neuroblastoma is a disease in which malignant (cancer) cells form in nerve tissue of the adrenal gland, neck, chest, or spinal cord.

Ewing's Sarcoma is a rare cancerous tumor that occurs in the bone or close to the bone and that strikes children and adolescents. Ewing's Sarcoma is a group of four different types of cancer including of Ewing tumor of bone (Ewing sarcoma of bone), extraosseous Ewing tumors, primitive neuroectodermal tumors (peripheral neuroepithelioma), and Askin tumors. These tumors all come from the same type of stem cell.

Rhabdomyosarcoma is a rare and cancerous tumor of the muscles that are attached to bones. This tumor is most commonly located in the structures of the head and neck, urogenital tract and arms or legs.

Thus, in one embodiment, the present invention provides a method of treating neuroblastoma comprising administering a therapeutically effective amount of a compound of formula (I) or a pharmaceutically acceptable salt thereof to a subject in need thereof.

In a further embodiment, the present invention provides a method of treating Ewing's Sarcoma comprising administering a therapeutically effective amount of a compound of formula (I) or a pharmaceutically acceptable salt thereof to a subject in need thereof.

In a further embodiment, the present invention provides a method of treating rhabdomyosarcoma comprising administering a therapeutically effective amount of a compound of formula (I) or a pharmaceutically acceptable salt thereof to a subject in need thereof.

The compounds of formula (I), particularly COMPOUND I, may be administered in pharmaceutical compositions or medicaments suitable for enteral, such as oral or rectal, and parenteral administration to subjects, particularly mammals (warm-blooded animals) including humans, comprising a therapeutically effective amount of the compound of formula (I) or pharmaceutically acceptable salt thereof with one or more pharmaceutically acceptable carriers or diluents, especially suitable for enteral or parenteral application.

Pharmaceutical compositions or medicaments for the combination therapy for enteral or parenteral administration are, for example, those in unit dosage forms, such as sugarcoated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

Thus, the present invention further provides a pharmaceutical composition or medicament comprising the compound of formula (I) or a pharmaceutically acceptable salt thereof, and optionally at least one pharmaceutically acceptable carrier, for use in the treatment of cancer selected from the group consisting of neuroblastoma, Ewing's Sarcoma, or rhabdomyosarcoma. In a preferred embodiment, the compound of formula (I) is COMPOUND I. In a further preferred embodiment, the compound of formula (I) or a pharmaceutically acceptable salt thereof is administered in a therapeutically effective dose to a subject in need thereof.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

Acceptable excipients are non-toxic, aid administration, and do not adversely affect the therapeutic benefit of the compound of formula I. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like.

Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990). The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

Suitable pharmaceutical compositions may contain, for example, from about 0.1% to about 99.9%, preferably from about 1% to about 60%, of the active ingredient(s). The actual amount of the compound of formula (I) administered in accordance with the present invention will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

The compound of formula (I), particularly the compound (S)-Pyrrolidine-1,2-dicarboxylic acid 2-amide 1-({4-methyl-5-[2-(2,2,2-trifluoro-1,1-dimethyl-ethyl)-pyridin-4-yl]thiazol-2-yl}-amide), may be administered orally at daily dosages of from about 0.03 to about 100.0 mg/kg per body weight, e.g. about 0.03 to about 10.0 mg/kg per body weight. An indicated daily dosage in the larger mammal, e.g. humans, is in the range from about 0.5 mg to about 3 g, e.g. about 5 mg to about 1.5 g, conveniently administered, for example, in divided doses up to four times a day or in retard form. Suitable unit dosage forms for oral administration comprise from approximately 0.1 to about 500 mg, e.g. about 1.0 to about 500 mg active ingredient, about 10.0 to 400 mg active ingredient.

Unless mentioned otherwise, the compound of formula (I) is used in a dosage as either specified in the product information of a product comprising such PI3-kinase inhibitor for the treatment of a proliferative disorder, or, especially if such product information is not available, in a dosage which is determined in dose finding studies.

Suitable clinical studies in human patients are, for example, open label nonrandomized, studies in patients with cancers selected from the group consisting of neuroblastoma, Ewing's Sarcoma, or rhabdomyosarcoma. Such studies prove in particular superiority of the claimed method of treatment compared to treatments with one of the components of the treatment schedule alone. The beneficial effects on neuroblastoma, Ewing's Sarcoma, or rhabdomyosarcoma can be determined directly through the results of these studies (e.g. RFS or progression free survival—PFS) or by changes in the study design which are known as such to a person skilled in the art.

In a further embodiment, the present invention provides a method of treating cancer selected from the group consisting of neuroblastoma, Ewing's Sarcoma, or rhabdomyosarcoma comprising administering a therapeutically effective amount of the compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”) or a pharmaceutically acceptable salt thereof to a subject in need thereof.

In a further embodiment, the present invention provides a method of delaying progression of cancers selected from the group consisting of neuroblastoma, Ewing's Sarcoma, or rhabdomyosarcoma in a patient comprising administering a therapeutically effective amount of a compound of formula I, particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof to a subject in need thereof.

The present invention further relates to the use of the compound of formula (I) or a pharmaceutically acceptable salt thereof for the manufacture of a pharmaceutical composition or medicament for use in the treatment of cancers selected from the group consisting of neuroblastoma, Ewing's Sarcoma, or rhabdomyosarcoma. In a preferred embodiment, the compound of formula (I) is COMPOUND I.

In a further embodiment, the present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for the manufacture of a pharmaceutical composition or medicament for use in delaying the progression of cancers selected from the group consisting of neuroblastoma, Ewing's Sarcoma, or rhabdomyosarcoma in a patient in need thereof.

In a further embodiment, the present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}amide) (“COMPOUND I”), or a pharmaceutically acceptable salt thereof for the manufacture of a pharmaceutical composition or medicament for use in delaying the progression of neuroblastoma in a patient in need thereof.

In a further embodiment, the present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for the manufacture of a pharmaceutical composition or medicament for use in delaying the progression of Ewing's Sarcoma in a patient in need thereof.

In a further embodiment, the present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for the manufacture of a pharmaceutical composition or medicament for use in delaying the progression of rhabdomyosarcoma in a patient in need thereof.

The present invention further relates to the use of the compound of formula (I) or a pharmaceutically acceptable salt thereof in the treatment of cancers selected from the group consisting of neuroblastoma, Ewing's Sarcoma, or rhabdomyosarcoma. In a preferred embodiment, the compound of formula (I) is COMPOUND I.

The present invention further relates to the use of the compound of formula (I) or a pharmaceutically acceptable salt thereof in the treatment of neuroblastoma.

The present invention further relates to the use of the compound of formula (I) or a pharmaceutically acceptable salt thereof in the treatment of Ewing's Sarcoma.

The present invention further relates to the use of the compound of formula (I) or a pharmaceutically acceptable salt thereof in the treatment of rhabdomyosarcoma.

In a further embodiment, the present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for use in delaying the progression of cancers selected from the group consisting of neuroblastoma, Ewing's Sarcoma, or rhabdomyosarcoma in a patient in need thereof.

The present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for use in delaying the progression neuroblastoma in a patient in need thereof.

The present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}amide) (“COMPOUND I”), or a pharmaceutically acceptable salt thereof for use in delaying the progression of Ewing's Sarcoma in a patient in need thereof.

The present invention further relates to the use of the compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), or a pharmaceutically acceptable salt thereof for use in delaying the progression of rhabdomyosarcoma in a patient in need thereof.

The present invention further relates to a compound of formula (I) or pharmaceutically acceptable salt thereof for use in the treatment of cancers selected from the group consisting of neuroblastoma, Ewing's Sarcoma, or rhabdomyosarcoma.

The present invention further relates to a compound of formula (I) or pharmaceutically acceptable salt thereof for use in the treatment of neuroblastoma.

The present invention further relates to a compound of formula (I) or pharmaceutically acceptable salt thereof for use in the treatment of Ewing's Sarcoma.

The present invention further relates to a compound of formula (I) or pharmaceutically acceptable salt thereof for use in the treatment of rhabdomyosarcoma.

In a further embodiment, the present invention relates to a compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), for use in delaying the progression of cancers selected from the group consisting of neuroblastoma, Ewing's Sarcoma, or rhabdomyosarcoma in a patient in need thereof.

The present invention relates to a compound of formula (I), particularly compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (“COMPOUND I”), for use in delaying the progression of neuroblastoma in a patient in need thereof.

The present invention relates to a compound of formula (I), particularly compound (S)-Pyrrolidine-1,2-dicarboxylic acid 2-amide 1-{4-methyl-5-[2-(2,2,2-trifiuoro-1,1-dimethyl-ethyl)-pyridin-4-yl]-thiazol-2-yl}-amide) (“COMPOUND I”), for use in delaying the progression of Ewing's Sarcoma in a patient in need thereof.

The present invention relates to a compound of formula (I), particularly compound (S)-Pyrrolidine-1,2-dicarboxylic acid 2-amide 1-({4-methyl-5-[2-(2,2,2-trifiuoro-1,1-dimethyl-ethyl)-pyridin-4-yl]-thiazol-2-yl}-amide) (“COMPOUND I”), for use in delaying the progression of rhabdomyosarcoma in a patient in need thereof.

The following Example illustrates the invention described above, but is not, however, intended to limit the scope of the invention in any way. Other test models known as such to the person skilled in the pertinent art can also determine the beneficial effects of the claimed invention.

In the following Examples, the following General Procedure is used to assess the sensitivity of specific neuroblastoma cell lines to COMPOUND I.

### General Procedure:

### Materials

Cell Culture:

Cell lines are obtained from commercial sources including ATCC and DSMZ. All cell lines tested are cultured in RPMI or DMEM plus 10% FBS (Invitrogen) as supplier recommended. Cell lines are cultured in T-175 or 3 layer T-175 “triple” flasks using standard tissue culture techniques that are frequently carried out with a robot (CompacT—The Automation Partnership). All cell lines are thawed from frozen stock, grown at 37 C 5% CO2. Adherent lines are dislodged using TrypLE (Invitrogen), are expanded through at least 1 passage of 1:3 dilution and usually 2 to 3 passages before they are added to assay micro-titer plates. Cell count and viability are measured using Trypan dye exclusion with a ViCell counter (Beckman-Coulture). All cell lines are tested for and shown to be free of Mycoplasma using a PCR detection (http://www.radil.missouri.edu).

Compound Preparation:

(S)-Pyrrolidine-1,2-dicarboxylic acid 2-amide 1-({4-methyl-5-[2-(2,2,2-trifiuoro-1,1-dimethyl-ethyl)-pyridin-4-yl]-thiazol-2-yl}-amide) (“COMPOUND I”) is dissolved in 90% DMSO/10% water at 2 mM and stored at −20° C. for long term storage and at room temperature for days at a time. The solutions are arrayed in microtiter plates and are serially diluted 3.16 fold seven additional times yielding a concentration range of 2 mM to 636 nM. Purity of the small molecules and solutions is checked using standard LC-Mass Spectroscopy checking UV adsorption and mass of the major UV peak.

### Assay Automation

All assays are automated and are performed with an ultra-high throughput screening system built by the Genomics Institute of the Novartis Research Foundation (GNF Systems; http://www.gnfsystems.com/). Cell lines are dispensed into Tissue culture treated 1536 well plates with a final volume of 5 uL and a concentration of 250 cells per well. Cells are allowed to adhere and begin growth for twelve to twenty-four hours; small molecules are then transferred to the cells using slotted pins (V&P Scientific, http://www.vp-scientific.com/index.html) calibrated to deliver 20 nL of material. This results in a final small molecule concentration range of 8 uM to 2.5 nM and a final DMSO concentration of just under 0.4%. The cell—small molecule mixture is incubated for 72 to 84 hours. Cell Titer Glo (Promega), which measures the amount of ATP in the well, is added and luminescence is read on a ViewLux plate reader (Perkin Elmer). On all plates, wells containing vehicle only and the positive control MG132 at 1 μM, a proteosome inhibitor, are included. Raw values are percent Normalized on a plate by plate basis such that 0% is equivalent to the median of vehicle wells and—100% equivalents to the median of the positive control. The Normalized data is further corrected using proprietary surface pattern model to remove edge and region effects (in Helios). All data is analyzed and stored in Helios data systems and subsequently loaded to the Avalon and Magma databases.

### Inflection Point (EC₅₀) and Amax Determination:

Cell line dose response curves are represented by a Hill sigmoidal function with a maximal effect level (Amax) curvature inflection point (EC₅₀).

### Statistical Analysis:

Statistical analysis of the associations between various lineage/genetic/pathway factors with COMPOUND I pharmacological sensitivity is carried out using the Fisher's exact test.

### Sensitivity Analysis:

To determine the sensitivity of various cancer cell lines to COMPOUND I, the effects of increasing doses of the compound on cell growth are examined in an extensive cancer cell line panel. Cells are plated in 1536-well micro titer plates and incubated for 72 hours in concentrations of COMPOUND I ranging in 3 fold-increments from 0.0025 to 8 uM. Following this incubation relative cell numbers are determined using cell titer glo and eight-point dose response curves across 594 cell lines are generated. EC₅₀ (inflection point) and Amax (maximum observed responses) are determined based on those curves, and the combination of the two parameters are used in this report to evaluate compound potency.

To define the population of the responsive cell lines for purposes of these experiments, the sensitivity of a cell line to COMPOUND I is defined by the cuttoff of EC₅₀ of less than 6 μmol/L and Amax of less than −35%. EC₅₀ is chosen over IC₅₀ based on the observation that many cell lines display partial responses to COMPOUND I but, in some cases, never reach 50% reduction of total cell numbers (which is the definition of an IC₅₀) yet the responses in many cases are robust and reliable. This suggests that PIK3CA signal is necessary but may not be sufficient in those models as single agent to induce complete tumor regression. Amax of <−35% is selected to filter out cell lines that the maximum responses are too shallow and are difficult to differentiate from noises.

### Example 1

### Neuroblastoma Cell Line Sensitivity to Compound I

To assess the sensitivity of neuroblastoma cell lines to COMPOUND I, the General Procedure set forth above is followed. The following neuroblastoma cell lines are used: CHP-212, IMR-32, KELLY, SK-N-SH, KP-N-SI9s (having KRAS mutation), SIMA, SK-N-AS, SK-N-BE(2), SK-N-DZ, and SK-N-FI. The CHP-212, KELLY, SK-N-SH, and SK-N-AS cell lines are normal for amplified ErbB2, normal for amplified PIK3CA, wildtype KRAS, wild type PTEN, wild type PIK3CA, wild type EGFR, wild type BRAF. The IMR-32 and SK-N-BE(2) cell lines are normal for amplified ErbB2 and normal for amplified PIK3CA. The SIMA, SK-N-DZ, and SK-N-FI cell fines are normal for amplified ErbB2, normal for amplified PIK3CA, wildtype KRAS, wild type PTEN, and wild type PIK3CA. The KP-N-SI9s cell line is having a KRAS mutation and normal for amplified ErbB2, normal for amplified PIK3CA, wild type PTEN, wild type PIK3CA, wild type EGFR, wild type BRAF.

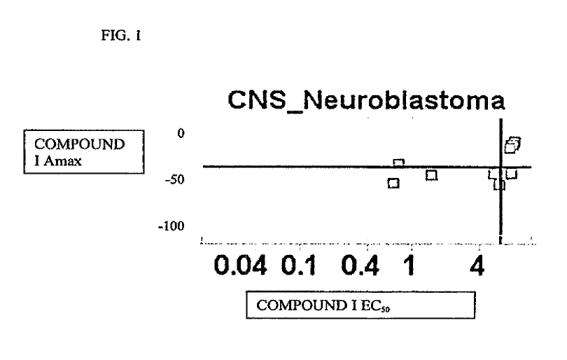

The pharmacological profiling results are summarized in Table 1 and are displayed as a Two-Tailed Fisher's Exact Test in FIG. 1. The Two-Tailed Fisher's Exact Test provides a comparison of Amax (% inhibition) of COMPOUND I to EC₅₀ of COMPOUND I.

TABLE 1

| | | COM- | COM- | |
|---|---|---|---|---|
| | | POUND I | POUND I | |
| Cell Name | Lineage_Detailed | EC₅₀ | Amax | Results |
| CHP-212 | CNS_Neuroblastoma | 0.8 | −30.9 | |
| IMR-32 | CNS_Neuroblastoma | 8.0 | −8.9 | |
| KELLY | CNS_Neuroblastoma | 8.0 | −4.3 | |
| SK-N-SH | CNS_Neuroblastoma | 1.6 | −44.6 | Sensitive |
| KP-N-SI9s | CNS_Neuroblastoma | 0.6 | −54.2 | Sensitive |
| SIMA | CNS_Neuroblastoma | 8.0 | −42.0 | |
| SK-N-AS | CNS_Neuroblastoma | 8.0 | −11.6 | |
| SK-N-BE(2) | CNS_Neuroblastoma | 8.0 | −8.7 | |
| SK-N-DZ | CNS_Neuroblastoma | 5.3 | −42.4 | Sensitive |
| SK-N-FI | CNS_Neuroblastoma | 6.1 | −55.6 | |

The results demonstrate that the SK-N-SH, KP-N-SI9s, and SK-N-DZ neuroblastoma cell lines are sensitive to COMPOUND I. Based upon these results, it can be concluded that COMPOUND I may be used for the treatment of neuroblastoma.

### Example 2

### Ewing's Sarcoma Cell Line Sensitivity to Compound I

To assess the sensitivity of Ewing's Sarcoma cell lines to COMPOUND I, the General Procedure set forth above is followed. The following Ewing's Sarcoma cell lines are used: Hs 822.T, MHH-ES-1 (having amplified PIK3CA), SK-ES-1, and TC-71. The MHH-ES-1 is having amplified PIK3CA and normal for amplified ErbB2, wildtype KRAS, wild type PTEN, wild type PIK3CA, wild type EGFR, wild type BRAF. The SK-ES-1 cell line is normal for amplified ErbB2, normal for PIK3CA, wildtype KRAS, wild type PTEN, wild type PIK3CA, wild type EGFR, wild type BRAF. The TC-71 cell line is normal for amplified ErbB2 and normal for PIK3CA.

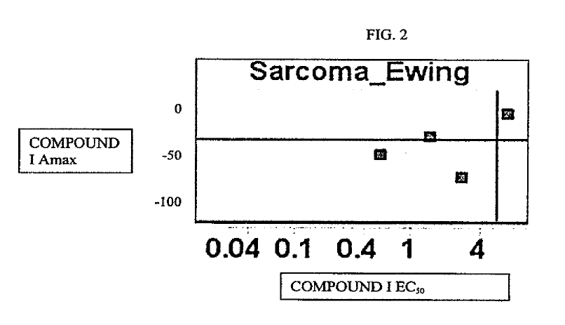

The pharmacological profiling results are summarized in Table 2 and are displayed as a Two-Tailed Fisher's Exact Test in FIG. 2. The Two-Tailed Fisher's Exact Test provides a comparison of Amax (% inhibition) of COMPOUND I to EC₅₀ of COMPOUND I.

TABLE 2

| | | COM- | COM- | |
|---|---|---|---|---|
| | | POUND I | POUND I | |
| Cell Name | Lineage_Detailed | EC₅₀ | Amax | Results |
| Hs 822.T | Sarcoma_Ewing | 1.5 | −31.6 | Sensitive |
| MHH-ES-1 | Sarcoma_Ewing | 2.7 | −79.2 | Sensitive |
| SK-ES-1 | Sarcoma_Ewing | 0.5 | −51.7 | Sensitive |
| TC-71 | Sarcoma_Ewing | 8.0 | −5.4 | |

The results demonstrate that the HS 822.T, MHH-ES-1, and SK-ES-1 Ewing's Sarcoma cell lines are sensitive to COMPOUND I. Based upon these results, it can be concluded that COMPOUND I may be used for the treatment of Ewing's s.

### Example 3

### Rhabdomyosarcoma Cell Line Sensitivity to COMPOUND I

To assess the sensitivity of Rhabdomyosarcoma cell lines to COMPOUND I, the General Procedure set forth above is followed. The following rhabdomyosarcoma cell lines are used: G-401 (Rhabdoid), G-402 (Leiomyoblastoma), A-673, Hs 729, KYM-1, RD, RH-41, SJRH30, TE 125.T, and TE 617.T.

The G-401 cell line is normal for amplified ErbB2, normal for amplified PIK3CA, wildtype KRAS, wild type PTEN, wild type PIK3CA, wild type EGFR, and wild type BRAF. The G-402 and TE617.T cell lines are normal for amplified ErbB2, normal for amplified PIK3CA. The A673 cell line is mutated for EGFR and amplified ErbB2, normal for amplified PIK3CA, wildtype KRAS, wild type PTEN, wild type PIK3CA, wild type BRAF. The Hs729, RD, SJRH30, cell lines are normal for amplified ErbB2, normal for amplified PIK3CA, wildtype KRAS, wild type PTEN, wild type PIK3CA, wild type EGFR, wild type BRAF. The KYM-1 cell line is wildtype KRAS, wild type PTEN, wild type PIK3CA, wild type EGFR, wild type BRAF.

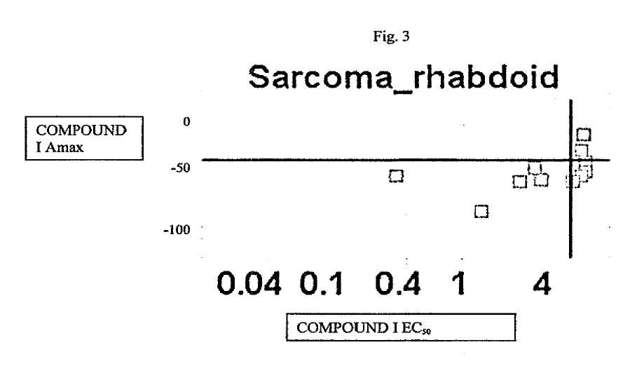

The pharmacological profiling results are summarized in Table 3 and are displayed as a Two-Tailed Fisher's Exact Test in FIG. 3. The Two-Tailed Fisher's Exact Test provides a comparison of Amax (% inhibition) of COMPOUND I to EC₅₀ of COMPOUND I.

TABLE 3

| | | COM- | COM- | |
|---|---|---|---|---|
| | | POUND I | POUND I | |
| Cell Name | Lineage_Detailed | EC₅₀ | Amax | Results |
| G-401 | Rhabdoid | 8.0 | −46.6 | |
| G-402 | Rhabdoid | 8.0 | −26.4 | |
| A-673 | Sarcoma_rhabdoid | 5.7 | −56.2 | Sensitive |
| Hs 729 | rhabdomyosarcoma | 8.0 | −10.2 | |
| KYM-1 | rhabdomyosarcoma | 8.0 | −38.0 | |
| RD | rhabdomyosarcoma | 3.3 | −43.2 | Sensitive |
| RH-41 | rhabdomyosarcoma | 1.6 | −85.4 | Sensitive |
| SJRH30 | rhabdomyosarcoma | 2.8 | −56.5 | Sensitive |
| TE 125.T | rhabdomyosarcoma | 8.0 | −49.8 | |
| TE 617.T | rhabdomyosarcoma | 3.8 | −55.2 | Sensitive |

The results demonstrate that the A673, RD, RH-41, SJRH30 and TE 617.T rhabdomyosarcoma cell lines are sensitive to COMPOUND I. Based upon these results, it can be concluded that COMPOUND I may be used for the treatment of rhabdomyosarcoma.

What is claimed is:

1. Method of treating neuroblastoma or Ewing's Sarcoma comprising administering a therapeutically effective amount of a compound (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) or a pharmaceutically acceptable salt thereof to a subject in need thereof.

2. Method according to claim 1, wherein the compound of formula (I) is administered in a daily dose between 0.1 to about 500 mg.
